(19) United States
(12) Patent Application Publication
(10) Pub. No.: US 2023/0181234 A1
(43) Pub. Date: Jun. 15, 2023

(54) ELECTROSURGICAL INSTRUMENT

(71) Applicant: GYRUS MEDICAL LIMITED, Cardiff (GB)

(72) Inventor: James Alan DICKSON, Cardiff (GB)

(73) Assignee: GYRUS MEDICAL LIMITED, Cardiff (GB)

(21) Appl. No.: 18/079,422

(22) Filed: Dec. 12, 2022

Related U.S. Application Data
(60) Provisional application No. 63/289,900, filed on Dec. 15, 2021.

(51) Int. Cl. A61B 18/14

(52) U.S. Cl. CPC A61B 18/14; A61B 2018/00083; A61B 2018/1412

(57) ABSTRACT

An electrosurgical instrument end effector comprises a rotary shaver arrangement, and an active electrode for supplying radio-frequency (RF) power to a surgical site. The rotary shaver arrangement comprises a rotatable shaver blade, and a stator that partially surrounds the rotatable shaver blade. At least part of the stator is electrically non-conductive.

### TECHNICAL FIELD

The present invention relates an electrosurgical instruments. More specifically, the present invention relates to end effectors of electrosurgical instruments.

### BACKGROUND TO THE INVENTION AND PRIOR ART

Surgical instruments, including radio frequency (RF) electrosurgical instruments, have become widely used in surgical procedures where access to the surgical site is restricted to a narrow passage, for example, in minimally invasive “keyhole” surgeries.

A RF arthroscopic device may have an active electrode and a larger return electrode. The electrodes receive a RF power signal from a generator. The active electrode delivers the RF power to a surgical site to perform RF functionalities, such as tissue ablation and/or coagulation. The RF device may also have a rotary shaving arrangement including a stator and an internal rotating blade. The stator may be used as the return electrode.

The active electrode and the return electrode (e.g. the stator) may require electrical separation, e.g. via a ceramic insulator between the active electrode and the stator. The insulator increases the complexity of the device and reduces its reliability since a leak-proof joint must be formed between the stator and the insulator to maintain suction during shaving. The stator and the insulator are also both relatively complex and expensive to manufacture individually and assemble.

Furthermore, the RF shaver device includes suction pathways for clearing surgical sites of debris. One pathway may be provided through the active electrode and the insulator, and into a lumen defined by the interiors of the stator and the internal rotating blade. Such a structure can cause there to be multiple current paths between the active electrode to the return electrode (e.g. the stator), not all of which are desirable. The extra, undesirable current paths can cause a loss in electrical efficiency of the RF ablation/coagulation functions of the RF shaver device.

### SUMMARY OF THE INVENTION

The present invention relates to an end effector assembly for an electrosurgical instrument which has improved efficiency in radio-frequency (RF) functionalities and reduced complexity. In particular, an opposite sided RF shaver is provided. The RF shaver has a rotary shaver arrangement for performing mechanical shaving and/or cutting functions. The rotary shaver arrangement has a rotatable shaver blade and a stator that partially surrounds the rotatable shaver blade to form a cutting window. In use, the rotatable shaver blade rotates within the stator to perform shaving and/or cutting functions at the cutting window. The RF shaver also has an electrode assembly including an active electrode. The stator is made from a conductive material (e.g. a metal, such as steel) and acts as a larger return electrode of the electrode assembly. The electrodes receive a RF power signal from a generator. Current conducts between the active electrode and the stator and an RF electrical field is formed at the active electrode. The active electrode therefore supplies the RF power to a surgical site to perform RF functions at the surgical site. RF functions can include tissue ablation and/or coagulation functionalities. The RF shaver can also comprise an outer shaft that is electrically connected to the stator, which forms part of the electrical path between the stator and the generator.

In opposite sided RF shaver arrangements, there can be multiple current paths between the active electrode and the stator (i.e. the return electrode). The optimum current path between the active electrode and the stator is between the active electrode and the external surface of the stator over an exterior of the RF shaver. This optimum current path creates the tissue ablation and/or coagulation effect on surrounding external target tissue. However, there can exist less optimum current paths, such as between the active electrode and an internal surface of the stator. This “internal” current path occurs, for example, due to the structural arrangement of the opposite sided RF shaver. In particular, the opposite sided RF shaver has a fluid channel extending through the active electrode to a lumen defined by the interiors of the rotatable shaver blade and the stator. In use, the lumen is connected to a pump and carries fluid from the surgical site away from the end effector. Consequently, there are also internal current paths from the active electrode to the inner surface of the stator, via the fluid channel. The internal current paths are typically not useful for the tissue ablation and/or coagulation effects. Therefore the internal current paths can causes a loss of efficiency in the RF functionality of the RF shaver, e.g. due to additional power losses in the device.

The present invention is able to diminish or eliminate the internal current paths by coating the stator in a non-conductive coating. In particular, the internal surfaces of the stator are coated in the non-conductive coating. Therefore, the internal conductive paths to the internal surfaces of the stator are avoided. At least part of the external surface of the stator remains uncoated. As such, the original conductive material of the stator remains exposed, thus forming the return electrode. Therefore, the optimal external return path is maintained for performing RF functions, such as tissue ablation and/or coagulation. However, in some examples, the stator can be entirely coated in the non-conductive coating. This may stop all current paths from the active electrode to the stator. Instead, the outer shaft can be used as the return electrode since it is conductive. Therefore, the internal current paths to the stator are avoided, whilst still providing an external current path from the active electrode to the outer shaft to enable the RF functions. In any event, the efficiency of the RF shaver (e.g. the electrical efficiency) is improved.

The present invention also leads to further improvements in the RF shaver device, including: reduced friction between the rotatable shaver blade and the stator leading to less wear and better mechanical efficiency; reducing the likelihood of internal fire-up of the RF device; and reducing the number of components and the complexity of the device. The complexity of the device is reduced because the ceramic insulator that is usually positioned in between the active electrode and the stator is not required, because the interface between the stator and active electrode will already be insulated by virtue of the non-conductive coating. This further removes the need to form a seal between a ceramic insulator and the stator, thus reducing the complexity of the assembly and improving its reliability.

According to a first aspect of the present disclosure, there is provided an end effector for an electrosurgical instrument, comprising a rotary shaver arrangement and an active electrode for supplying radio-frequency (RF) power to a surgical site. The rotary shaver arrangement comprises a rotatable shaver blade, and a stator that partially surrounds the rotatable shaver blade, wherein at least part of the stator is non-conductive.

Advantageously, by making at least part of the stator non-conductive, undesired current paths between the active electrode and the stator can be avoided, thus improving the efficiency of the RF functionalities of the end effector (e.g. the electrical efficiency). Furthermore, the active electrode can be placed directly onto a non-conductive region of the stator, thus removing the need for a ceramic/polymer insulating component and therefore reducing the complexity and improving the reliability of the end effector.

In some embodiments, the active electrode comprises an aperture which provides access to a lumen for carrying fluid from the surgical site, the lumen is at least in part defined by an inner surface of the stator, and at least the inner surface of the stator is non-conductive.

Advantageously, the end effector is able to perform suctioning to clear the surgical site of debris. However, undesired current paths reaching the inner surface of the stator via the suction aperture may be undesired, because they use up electrical energy but do not contribute to the main RF functionalities of the device (tissue ablation and coagulation). By making the inner surface of the stator non-conductive, undesired current paths reaching the inner surface of the stator via the suction aperture can be avoided, thus improving the device efficiency.

In some embodiments, the stator comprises an outer surface, and at least part of the outer surface of the stator is conductive to form a return electrode of the electrode assembly.

Advantageously, a desired current path from the active electrode to the stator can be provided. In particular, an external current path from the active electrode to the outer or exterior surface of the stator can be provided, whilst avoiding the undesired current paths (e.g. the internal current paths). The external current path is desired because it contributes to the main RF functionalities of the device (e.g. tissue ablation and/or coagulation).

In some embodiments, the active electrode is coupled to a coupling region of the stator, the coupling region being non-conductive. Additionally or alternatively, there is no ceramic or polymer insulating element in between the active electrode and the stator.

Advantageously, the complexity of the end effector is reduced in comparison to opposite sided RF shavers that include a ceramic/polymer insulating component. In particular, the end effector of the present invention can have fewer components, and the need to form a seal between an insulating component and the stator is avoided thus improving the reliability of the device also.

In other embodiments, the end effector can include an insulating element in between the active electrode and the stator.

As such, instead of providing a non-conductive coupling region on the stator, an insulating component can be provided to insulate the active electrode and the stator from direct electrical contact.

In some embodiments, the end effector includes a retention means for retaining the active electrode to the coupling region or to the insulating element.

Advantageously, the active electrode can be secured to the end effector. In alternative embodiments, the active electrode and the retainer can form a single component (i.e. a one piece tip).

In some embodiments, the stator comprises: a substrate (or core) formed of a conductive material; and a non-conductive coating provided over at least part of the substrate.

Advantageously, different portions of the stator can be made conductive or non-conductive by selectively choosing which areas of the conductive substrate material are coated in the non-conductive coating. Moreover, the non-conductive regions of the stator can be provided by a relatively simple process of coating the stator.

In some embodiments, the inner surface of the stator is non-conductive by means of the non-conductive coating.

In some embodiments, at least part of the outer surface of the stator is conductive by an absence of the non-conductive coating.

The stator may be natively formed of a conductive material. As such, the conductive part of the outer surface can be formed simply by leaving the conductive area uncoated. For example, the conductive area can be masked prior to a coating process. Then, the coating process can be performed. Afterwards, the mask can be removed, and therefore the conductive area formed by the conductive material of the stator will remain.

In some embodiments, the coupling region is non-conductive by means of the non-conductive coating.

Advantageously, the active electrode can be coupled directly to a non-conductive surface or surfaces of the stator without requiring an insulating element, since the non-conductive coating can provide the insulation barrier between the stator and the active electrode.

In some embodiments, the non-conductive coating is a diamond-like carbon (DLC).

Advantageously, DLC is able to provide the necessary electrical insulation at a relatively thin coating. Furthermore, DLC coating processes have been found suitable for coating the stator of the present invention.

In some embodiments, the substrate of the stator is formed from a metal, and preferably wherein the metal is any one of copper, stainless steel, tungsten or an alloy of tungsten and platinum.

As such, the substrate of the stator can advantageously be formed of various different materials.

In some embodiments, the stator comprises an outer surface, wherein the outer surface is non-conductive. Optionally, the entire surface of the stator is non-conductive.

Furthermore, in some embodiments, the end effector comprises an outer shaft, wherein the outer shaft is electrically conductive to form a return electrode of the electrode assembly.

In this embodiment, the stator does not form a return electrode and therefore does not form part of an electrical current path to the generator. Instead, a current path is provided between the active electrode and the conductive outer shaft for enabling RF functionalities (e.g. tissue ablation and coagulation) at the active tip. Advantageously, the end effector avoids the undesired internal current paths between the active electrode and the stator, whilst also being easier to manufacture. In particular, it is not necessary to add and remove a mask when coating the stator with the non-conductive coating. It will also be appreciated that in such an embodiment, the stator can instead be formed of a non-conductive material that does not require coating to make its surface non-conductive, such as a ceramic or polymer.

In some embodiments, the outer shaft is at least partially covered in an insulating material. The insulating material may be a heat-shrink.

Advantageously, the outer shaft is insulated from the environment whilst protecting a user from the return current in the outer shaft. It will be appreciated that when the outer shaft forms the return electrode, an area on the external surface of the outer shaft proximal to the active electrode may remain uncovered by the insulating material, in order to form the return electrode.

In some embodiments, the inner surface and the outer surface of the stator are non-conductive by means of a non-conductive coating, optionally wherein the entire surface of the stator is coated in the non-conductive coating.

In some embodiments, the active electrode is formed from a metal, and preferably wherein the metal is any one of copper, stainless steel, tungsten or an alloy of tungsten and platinum.

The outer shaft may be formed from a metal, and preferably wherein the metal is any one of copper, stainless steel, tungsten or an alloy of tungsten and platinum.

Advantageously, the conductive components of the end effector can be formed of a variety of conductive materials available.

In some embodiments, the rotatable shaver blade is formed from a ceramic or insulated steel.

Advantageously, the inner rotatable shaver blade of the rotary shaver arrangement does not conduct an internal current from the aperture of the active electrode. Therefore, undesired internal current paths from the active electrode to the stator are more robustly avoided.

In some embodiments, the stator partially surrounds the rotatable shaver blade so to form a shaving window of the rotary shaver arrangement, and wherein the active electrode is coupled to the rotary shaver arrangement on an opposite side to the shaving window.

As such, the advantages of the present invention are apparent for opposite sided end effectors.

In a second aspect of the present disclosure, there is provided an electrosurgical instrument, comprising: a hand-piece; one or more user-operable buttons on the handpiece for operably controlling the instrument, and an operative shaft, having RF electrical connections, and drive componentry for an end effector, the electrosurgical instrument further comprising an end effector according to the first aspect, the rotary shaver arrangement of the end effector being operably connected to the drive componentry to drive the rotary shaver arrangement to operate in use, and the active electrode being connected to at least one of the RF electrical connections.

In a third aspect of the present disclosure, there is provided an electrosurgical system, comprising: an RF electrosurgical generator; a suction source; and an electrosurgical instrument according to the second aspect, the arrangement being such that in use the RF electrosurgical generator supplies an RF signal having a coagulation or ablation waveform via the RF electrical connections to the active electrode.

### BRIEF DESCRIPTION OF THE DRAWINGS

Embodiments of the invention will now be further described by way of example only and with reference to the accompanying drawings, wherein like reference numerals refer to like parts, and wherein:

FIG. 1 is an example of the electrosurgical instrument system comprising an electrosurgical instrument according to the present invention;

FIG. 2 is an example of an RF electrosurgical instrument;

FIG. 3 is a cross-sectional perspective diagram of an opposite sided RF shaver, with a RF insulator;

FIG. 4A is a cross-sectional diagram of an opposite sided RF shaver with a stator with a non-conductive coating and with a RF insulator;

FIG. 4B is a cross-sectional perspective diagram of an opposite sided RF shaver with a stator with a non-conductive coating and with a RF insulator;

FIG. 4C is a perspective diagram of an opposite sided RF shaver with a stator with a non-conductive coating and with a RF insulator;

FIG. 4D is another perspective diagram of an opposite sided RF shaver with a stator with a non-conductive coating and with a RF insulator;

FIG. 5A is a cross-sectional perspective diagram of an opposite sided RF shaver, with a stator with a non-conductive coating and without a RF insulator;

FIG. 5B is a perspective view of an opposite sided RF shaver, with a stator with a non-conductive coating and without a RF insulator; and

FIGS. 6A-6B show perspective views of another opposite sided RF shaver, with a non-conductive stator and without a RF insulator.

### DETAILED DESCRIPTION OF THE EMBODIMENTS

Embodiments of the present invention involve a modification of an opposite sided RF shaver device. In particular, embodiments involve a modification of the stator component of the end effector. The stator is coated in a non-conductive coating, such as a diamond-like carbon (DLC). In particular, the internal surfaces of the stator are coated in order to avoid internal current paths between the active electrode and the inner surfaces of the stator, e.g. via the fluid channel through the active electrode into the lumen defined at least by the interior of the stator. However, at least part of the external surface of the stator remains uncoated thus exposing the core conductive material of the stator. The uncoated region forms the return electrode and provides the desired current path between the active electrode and the outer surface of the stator. Alternatively, the outer surface of the stator can be entirely coated with the non-conductive coating so that the stator cannot conduct current from the active electrode, and instead an outer shaft of the device can form the return electrode to provide the desired external current path for providing the RF functionalities.

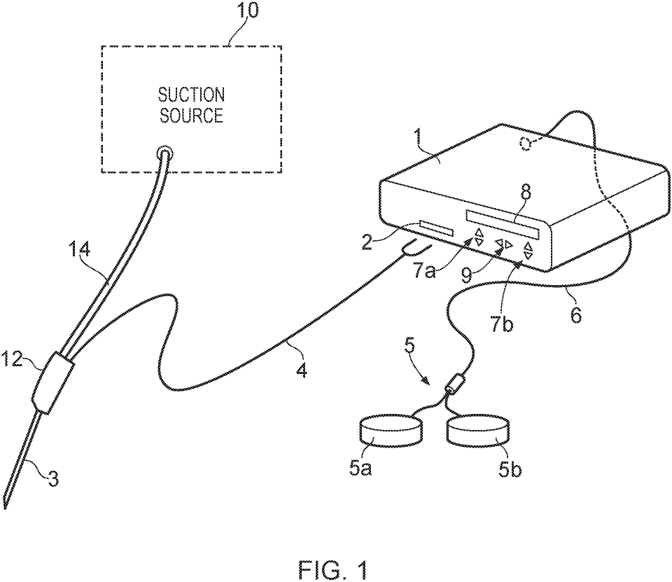

FIG. 1 shows an electrosurgical apparatus including an electrosurgical generator 1 having an output socket 2 that provides a radio frequency (RF) output (e.g. a RF power signal), via a connection cord 4, to an electrosurgical instrument 3. The instrument 3 has a suction tube 14 which is connected to a suction source 10. Activation of the generator 1 may be performed from the instrument 3 via a handswitch (not shown) on the instrument 3, or by means of a footswitch unit 5 connected separately to the rear of the generator 1 by a footswitch connection cord 6. In the illustrated embodiment, the footswitch unit 5 has two footswitches 5a and 5b for selecting a coagulation mode or a cutting or vaporisation (ablation) mode of the generator 1 respectively. The generator front panel has push buttons 7a and 7b for respectively setting ablation (cutting) or coagulation power levels, which are indicated in a display 8. Push buttons 9 are provided as an alternative means for selection between the ablation (cutting) and coagulation modes.

The electrosurgical instrument 3 can be an opposite (or dual) sided RF shaver device. In particular, the main RF functionality and the mechanical shaving/cutting functionality of the instrument 3 can be provided on opposite sides of the distal end portion of the instrument 3. The structure of the RF side of the instrument 3 and the shaving side of the instrument 3 are described in more detail below.

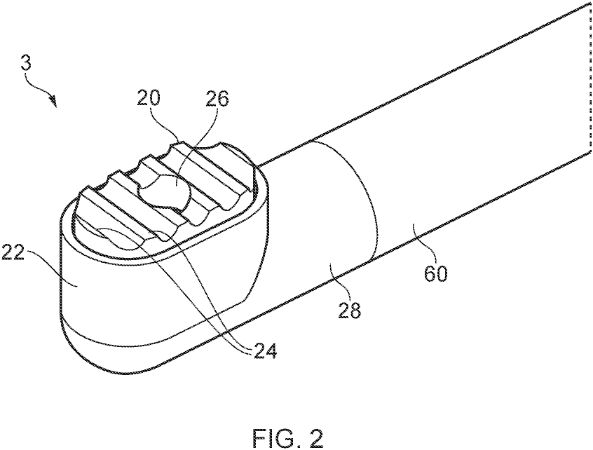

FIG. 2 shows an example a distal end portion of the electrosurgical instrument 3 in more detail. In particular, an RF side of the distal end portion of the electrosurgical instrument 3 is shown. The RF side of the electrosurgical instrument 3 includes an electrode assembly comprising an active electrode for tissue treatment (i.e. the “active tip”) 20, an insulator 22, and a return electrode 28. The active electrode 20 is received in the insulator 22. The insulator 22 is provided in between the active electrode 20 and the return electrode 28 to physically separate the active electrode 20 from the return electrode 28. The active electrode 20 and the (larger) return electrode 28 receive the RF power signal from the generator 1 (not shown). A current will conduct between the active 20 and the return 28 electrodes via a RF tracking path that traverses over the exterior of the distal end portion. As a result, the active electrode 20 will produce an external electric field in response to the RF power signal. The electric field is used to treat tissue at a surgical site in the proximity of the active tip 20. The RF power signal from the generator can have an ablation or coagulation waveform for providing tissue ablation or coagulation functionalities at the active tip 20, respectively. The active tip 20 is provided with projections or protrusions 24 to concentrate the electric field at those locations. The projections 24 also serve to create a small separation between the planar top surface of the active electrode 20 and the tissue to be treated at the surgical site. This allows conductive fluid to circulate over the planar surface, and avoids overheating of the electrode or the tissue.

The active tip 20 of the instrument is provided with a primary suction aperture 26, which is the opening to a primary fluid channel (not shown). The primary fluid channel extends to a lumen (not shown). The lumen extends through the return electrode 28 and an outer shaft 60 of the instrument 3. The lumen therefore connects the suction aperture 26 to the suction pump 10 (see FIG. 1) via the suction tube 14 to transport fluids from the active tip 20 to the pump 10. The lumen may also include means (e.g. one or more wire or conductive paths) for electrically connecting the active electrode 20 to the generator 1. The return electrode 28 may be connected to the generator 1 via the outer shaft 60 of the instrument 3. In particular, the return electrode 28 may be electrically connected to the outer shaft 60 of the instrument 3 and the outer shaft 60 may be electrically conductive. Alternatively, the shaft 60 may be integrally formed with the return electrode 28. In any case, the RF power signal can be delivered from the generator 1 to the active 20 and return 28 electrodes, via the cord 4, so that the active tip 20 can perform external RF functionalities such as tissue ablation and/or coagulation. The mechanical shaver/cutting side of the electrosurgical instrument 3 is not shown in FIG. 2.

The active electrode tip 20 is formed of an electrically conductive material. The electrically conductive material may be any material suitable for forming an active electrode tip 20, for example, a metal such as copper or a stainless steel, tungsten or an alloy of tungsten and platinum. The insulator 22 can be a ceramic insulator. Alternatively, the insulator 22 can be made from a polymer. The insulator 22 can otherwise be any other suitable material for providing an insulation from electrical contact.

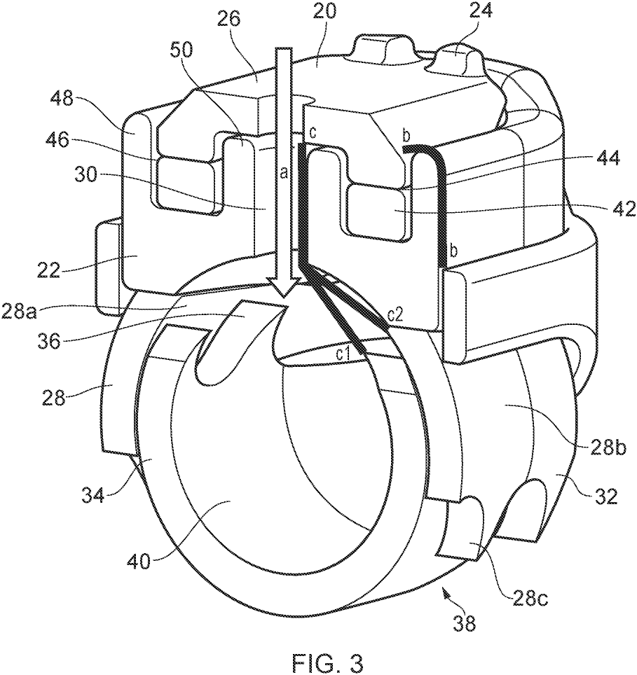

FIG. 3 shows a cross section of the distal end portion of the opposite sided RF shaver electrosurgical instrument 3. FIG. 3 shows both the RF side of the electrosurgical instrument 3 (top side) and the mechanical shaver/cutting side of the electrosurgical instrument 3 (bottom side).

The cross section of FIG. 3 shows the active tip 20 having the projections 24. The active tip 20 may be held in place in the insulator 22 by a retainer 42. Where the retainer 42 and the active tip 20 meet is referred to as the mating portion 44. Alternatively, the active tip 20 and the retainer 42 may be formed as a single component (a “one-piece tip”).

The cross section of FIG. 3 also shows the primary suction aperture 26, which is the opening to a primary fluid channel 30 extending to a lumen 40. The arrow a indicates the saline suction path during RF use (i.e. through the primary suction channel 30). The primary suction channel 30 extends from the surface of the active tip 20 through the active tip 20 and the insulating material 22 to the lumen 40. The opening of the primary suction channel 26 is located on the surface of the active tip 20 (“the primary suction aperture” 26). The primary suction aperture 26 may be located in the centre of the active tip 20, or offset to one side. The insulating material 22 comprises a recess 46 where the retainer 42 is positioned. The recess 46 is defined by outer 48 and inner 50 lips. On top of the retainer 42, the active tip 20 is held in place such that the active tip 20 extends above the outer lips 48 of the insulating material 22. In the case of a one-piece tip, the active tip 20 is positioned directly into the recess 46 of the insulting material 22. The primary suction channel 30 runs between the two inner lips 50 from the primary suction aperture 26 directly down to the lumen 40. The lumen 40 is connected to the suction source 10 via suction tubes 14 (as shown in FIG. 1).

The cross section of FIG. 3 also shows the return electrode 28 of the electrode assembly, which also acts as a stator of the rotary shaver arrangement. The stator 28 is connected to the bottom of the insulating component 22 such that the insulating component 22 insulates the active tip 20 from the stator 28. In particular, the insulating component 22 insulates the active tip 20 from direct electrical contact with the stator 28. The insulating material 22 may be, for example, a ceramic material such as alumina, zirconia toughened alumina (ZTA), yttria stabilized zirconia (YTZP) or the like.

The stator 28 has cutting teeth 32 which frame a cutting window 38 formed in a bottom of the stator 28. The instrument 3 also comprises an inner shaver blade 34 having cutting teeth 36. The stator 28 and the inner shaft 34 are concentrically arranged such that the cutting teeth 32 can also frame the cutting window 38. When the shaver component (i.e. the inner and the outer blades) is in use, the inner shaver blade 34 rotates such that the inner 36 and the outer 32 teeth cut tissue. In FIG. 3, the shaver component is not in use, and the cutting window 38 is closed by an outer surface of the inner shaver blade 34. The inner shaver blade 34 may receive power from the generator 1 to rotate, for example via the cord 4 and/or drive componentry or otherwise. The interior of the stator 28 and/or the inner shaver blade 34 defines the lumen 40 which carries fluid from the active tip 20. The stator 28 is made of an electrically conductive material, for example a metal such as copper, stainless steel, tungsten or an alloy of tungsten and platinum.

The stator 28 is the return electrode of the electrode assembly and is therefore electrically connected to the generator 1, for example via the cord 4, to receive RF power from the generator 1. The stator 28 may be electrically connected to the generator 1 via the outer shaft 60 (not shown in FIG. 3). The active tip 20 may also be electrically connected to the generator 1 via the cord 4 to receive the RF power signal from the generator 1. When RF power is supplied to the active tip 20 and stator 28, current will conduct between the active tip 20 and the stator 28 even though they are physically separated by the insulator 20. Current may conduct between the active tip 20 and the stator 28 via multiple RF tracking paths. The line bb indicates a preferred shorter RF tracking path between the active and return electrodes (i.e. the electrical current path between the active and the return electrodes). In particular, it is preferred that the RF tracking path is external to the instrument 3, i.e. between the active tip 20 and the exterior surfaces 28b of the stator 28 over an exterior of the insulator 22 as indicated by the line bb. However, the line cc2 indicates a further possible longer and less preferred RF tracking path from the active tip 20, through the primary suction channel 30, to the inner surfaces 28a of the stator 28. Therefore, during use of the instrument 3, it may be the case that some current will track internally to the inner surface 28a of the stator 28, e.g. via the line cc2, in addition to current tracking externally to the outer surface 28b of the stator 28, e.g. via the line bb. This can result in a loss of efficiency of the RF functionality at the active tip 20, because the internal current is typically not useful for providing the main RF functions of the RF shaver.

The inner blade 34 can also be part of a coincidental current path by virtue of being conductive and in close contact with the outer blade 28. In particular, the line cc1 indicates a further possible longer and less preferred tracking path, through the primary suction channel 30 to inner blade 34 edge, and eventually to the inner surfaces 28a of the stator 28. This can also lead to inefficiency in the RF functionality. However, in some examples, the inner blade 36 can be made from an insulated material (e.g. insulated steel, or fully ceramic). In this case, the inner blade 34 does not conduct current, and therefore the less preferred tracking path cc1 is diminished. Otherwise, the inner blade 34 can be a conductive material, e.g. a metal, such as copper, stainless steel, tungsten or an alloy of tungsten and platinum.

In any case, there remains the problem of inefficiencies caused by the internal tracking paths between the active electrode 20 and the stator 28 through the primary suction channel 30, e.g. via the path cc2 and the path cc1 if the inner blade 34 is a conductive material.

Furthermore, in order to maintain suction during the use of the instrument 3, the insulator 22 and the stator 28 need to be assembled to form a leak-proof joint, which can be a manufacturing burden. Moreover, each of the insulator 22 and the stator 28 are individually complex and expensive to manufacture.

In accordance with an embodiment of the present invention, the stator 28 is partially coated in a non-conductive coating. In particular, at least the inner surfaces 28a of the stator 28 are coated with the non-conductive coating. Preferably, the entire inner surface 28a of the stator 28 is coated with the non-conductive coating. The edges 28c of the stator 28 can also be coated with the non-conductive coating. The non-conductive coating can be, for example, a diamond-like carbon (DLC). However, any other suitable non-conductive coating can be used. At least part of the exterior stator surface 28b is left uncoated. In some examples, the entire external surface 28b of the stator can be left uncoated. In other examples, only a section of the external surface 28b of the stator 28 is left uncoated. Consequently, the stator 28 is generally non-conductive by virtue of the non-conductive coating, apart from one or more areas on the external surface 28b that are left uncoated. The uncoated area(s) expose the original conductive material (i.e. the original conductive substrate) of the stator 28. The exposed uncoated area(s) of the stator 28 form the return electrode. Therefore the RF current path bb between the active tip 20 and the exterior of the stator 28 is provided. However, the current path cc2 (and cc1) is avoided, since the inner surfaces 28a of the stator 28 are non-conductive. As such, the efficiency of the instrument 3 is improved by partially coating the stator 28 in a non-conductive material. It will be appreciated that coating the inner surfaces 28a of the stator 28 is sufficient for achieving this advantage, and therefore it is not essential for the edges 28c or parts of the exterior surfaces 28b to be non-conductive.

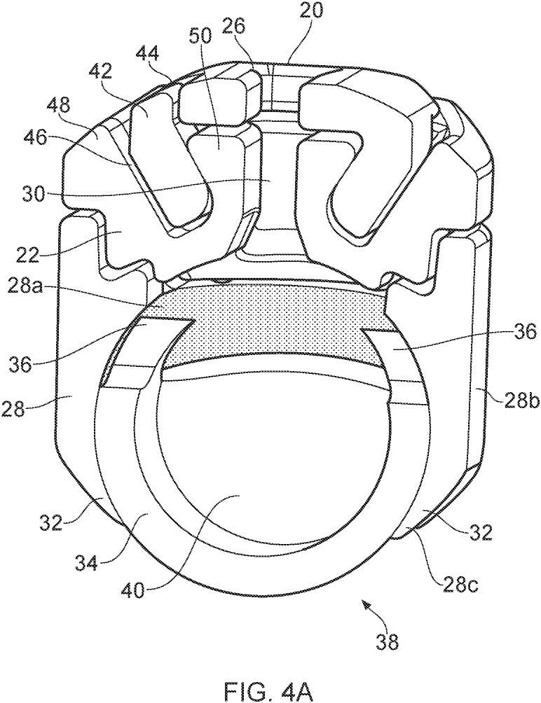

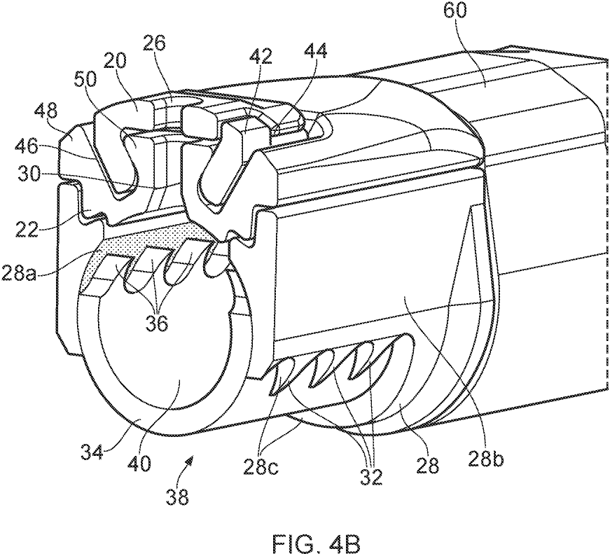

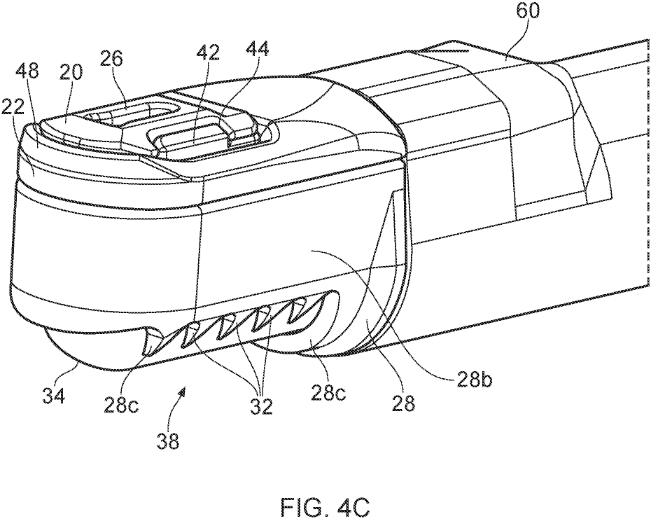

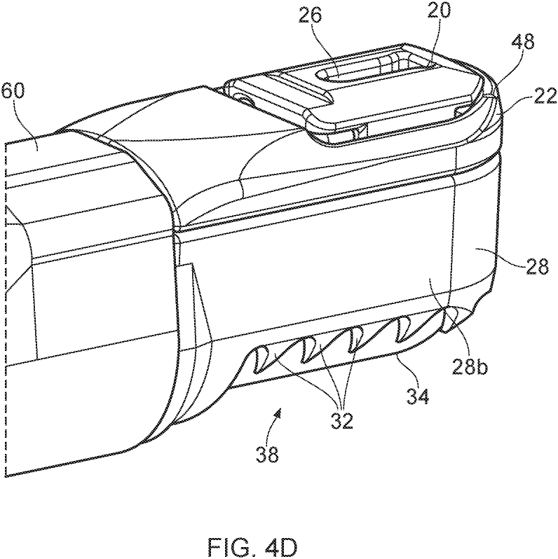

FIGS. 4A-4D show an example of a distal end portion of an electrosurgical instrument 3 having a stator 28 with a non-conductive coating, according to an embodiment of the present invention. The distal end portion of FIGS. 4A-4D differs from that of FIGS. 2 and 3 in that the inner surface 28a of the stator 28 is coated in a non-conductive material. The coated inner surface 28a is indicated by the shaded/textured region of the stator 28 in FIGS. 4A and 4B. The non-conductive material is a material that is not electrically conductive. The non-conductive material can be, for example, a diamond-like carbon (DLC). However, any other non-conductive material can be used. The rest of the stator 28 is not coated in the non-conductive material. In particular, the outer surface 28b of the stator 28 is not coated in the non-conductive material. Furthermore, the edges 28c of the stator 28 may also remain uncoated. The non-coated surfaces of the stator 28 are indicated by the absence of shading/texture in FIGS. 4A-4D.

The non-conductive coating can be applied to the inner surface 28a of the stator using any means available to the skilled person. The stator 28 is preferably coated before the stator 28 is assembled with the other components of the distal end portion. In one example, the stator 28 has a conductive substrate. The surfaces of the stator 28 that are to remain uncoated are masked, e.g. by a masking material. The non-conductive coating is then applied to the stator 28, e.g. to the entire stator 28. The masking material is then removed. Consequently, the coating remains on the inner surface 28a because that area was not masked. However, removing the masking material exposes the original conductive substrate material of the stator 28, which forms the return electrode.

Advantageously, the efficiency of the instrument 3 is improved by coating the inner surface 28a of stator 28 in a non-conductive material, where the less preferred current path cc2 (and cc1) occurs. Since the inner surface 28a does not conduct current, the current paths cc2 and cc1 do not occur. Moreover, since the inner surfaces 28a of the stator are coated, this can result in reduced friction between the inner blade 34 and the adjacent inner surfaces 28a of the stator 28. The reduced friction can result in reduced wear to the blade 34 and increased cutting efficiency of the instrument 3. Furthermore, the main electrical return path is only external to the stator 28, and therefore the likelihood of internal RF fire up within the device is reduced.

As shown in FIGS. 4A-4D, the retainer 42 in FIGS. 4A-4D may also differ to the retainer in FIG. 3. As such, different shapes and arrangements of the retainer 42 and the active tip 20 are possible. Furthermore as shown in FIGS. 4B-4D, the stator 28 is coupled to the outer shaft 60 which is also made of a conductive material such as a metal. The metal can be any one of copper, stainless steel, tungsten or an alloy of tungsten and platinum. The outer shaft 60 can form part of the electrical path between the stator 28 and the generator 1. The outer shaft 60 can optionally be coated or wrapped in an insulating material. Preferably, the outer shaft is wrapped in a heat shrink. Although not shown in FIGS. 4A-4D, the active tip 20 can be provided with projections or protrusions to concentrate the electric field at those locations, as described in connection with FIGS. 2 and 3.

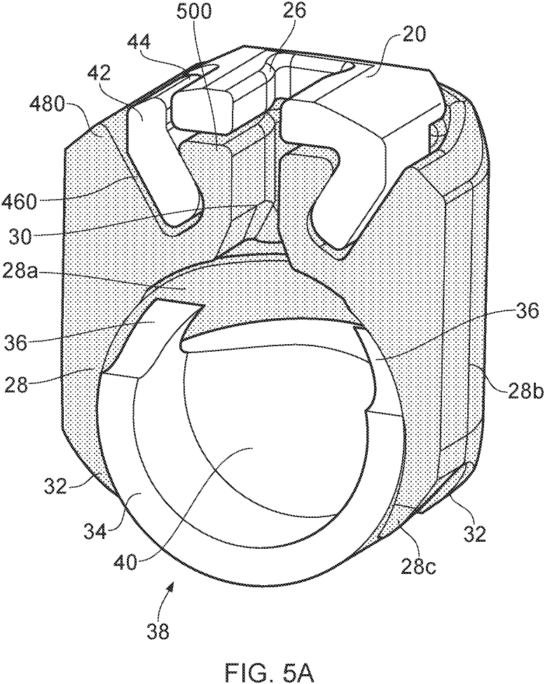

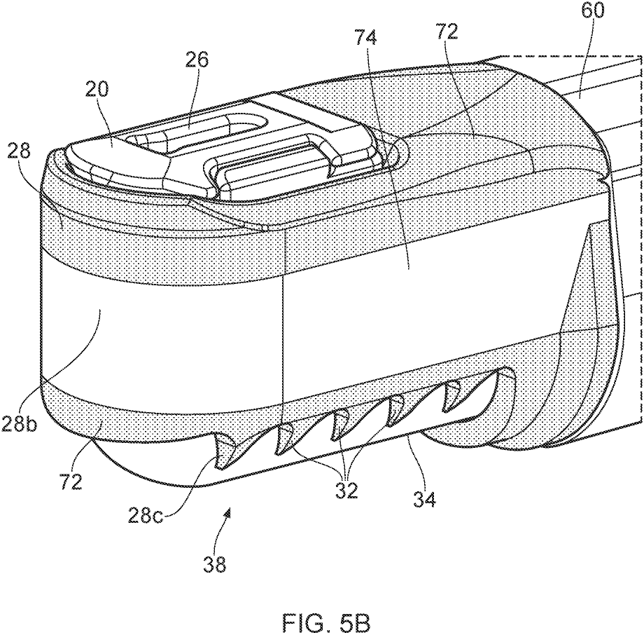

FIGS. 5A-5B show a further example of a distal end portion of an electrosurgical instrument 3 having a stator 28 with a non-conductive coating, according to an embodiment of the present invention. The distal end portion of FIGS. 5A-5B is similar to the distal end portion shown in FIGS. 4A-4D in that the inner surface 28a of the stator 28 is coated in the non-conductive material. However, as shown in FIG. 5B, the distal end portion in FIGS. 5A-5B differs in that the exterior surface 28b of the stator 28 comprises areas 72 and 74. The area 72 is coated in the non-conductive coating. The rest of the stator 28, including the edges 28c and the inner surface 28a are also coated in the non-conductive coating. The area 74 is left uncoated, exposing the original conductive material of the stator 28. In other words, the stator 28 is entirely coated in the non-conductive coating (indicated by the shaded/textured areas of the stator 28 in FIGS. 5A-5B) apart from the area 74 on the external surface 28b of the stator 28 (indicated by the absence of shading/texture in FIG. 5B). As such, the area 74 forms the return electrode of the instrument 3 to provide an electrical current path between the return electrode (stator) 28 and the generator 1, as described above. As shown in FIGS. 5A-5B, the stator 28 is coupled to the outer shaft 60 which is also made of a conductive material such as a metal. The metal can be any one of copper, stainless steel, tungsten or an alloy of tungsten and platinum. The outer shaft 60 can form part of the electrical current path between the return electrode and the generator 1. The outer shaft 60 can optionally be coated or wrapped in an insulating material. Preferably, the outer shaft is wrapped in a heat shrink. Although not shown in FIGS. 5A-5B, the active tip 20 can be provided with projections or protrusions to concentrate the electric field at those locations, as described in connection with FIGS. 2 and 3.

As such, the distal end portion shown in FIGS. 5A-5B achieves the same advantages as the one shown in FIGS. 4A-4D. For example the efficiency of the instrument 3 is improved by partially coating the stator 28 in a non-conductive material, in particular by coating the inner surfaces 28a of the stator 28 where the less preferred return path cc2 (and cc1) occurs. Moreover, since the inner surfaces 28a of the stator are coated, this can result in reduced friction between the inner blade 34 and the adjacent inner surfaces 28a of the stator 28. The reduced friction can result in reduced wear to the blade and increased cutting efficiency. Furthermore, since all of the internal surfaces of the stator 28 are coated, the main electrical path between the active tip 20 and the stator 28 is external to the stator 28, and therefore the likelihood of internal RF fire up within the device is reduced.

Optionally, as shown in FIGS. 5A-5B, the insulating component 22 shown in FIGS. 2, 3 and 4A-4D can be omitted. Instead, the stator 28 comprises a recess 460 where the retainer 42 is positioned. The recess 460 is defined by outer 480 and inner 500 lips. The active tip 20 is held in place by the retainer such that the active tip 20 extends above the outer lips 480 of the stator 28. However in the case of a one-piece tip (where the retainer 42 and the active tip 20 are formed of the same component), the active tip 20 is positioned directly into the recess 460 of the stator 28. Furthermore the primary suction channel 30 runs between the two inner lips 500 from the primary suction aperture 26 directly down to the lumen 40. Therefore, the active tip 20 can be coupled directly to stator 28 without a separate insulator, e.g. to the top side of the stator 28 on an opposite side to the shaving window 38.

The omission of the insulating element will mean that the active tip 20 directly mates or contacts with a surface of the stator 28. For example the active tip 20 may make contact with the surface in the recess 460 of the stator 28 where the active tip 20 is positioned. The surface of the stator 28 that contacts the active tip 20 may be referred to a coupling region of the stator 28. As discussed above, the stator 28 (including the coupling region) is coated in a non-conductive coating, apart from at the area 74 which is left uncoated. The non-conductive coating therefore provides the electrical insulation between the active tip 20 and the conductive substrate of the stator 28 even when the active tip 20 is coupled to and in direct contact with the stator 28. As such, the insulating component 22 is no longer required. This reduces the number of components required in the instrument 3, and also removes the requirement to form a complex seal between the insulator 22 and the stator 28. Consequently, the complexity and mechanical reliability of the instrument 3 is reduced and increased, respectively.

The stator 28 can be coated during a manufacturing process as follows. The stator 28 can initially have a conductive substrate. The area 74 to be left uncoated can be masked. Then, the entire stator 28 can be coated. When the mask is removed, the area 74 will be uncoated, exposing the original conductive substrate material of the stator 28 and thus forming the return electrode. The partially coated stator 28 can then be assembled with the other components to form the instrument 3. It will be appreciated that any other techniques known to the skilled person may be used to provide the partially coated stator 28.

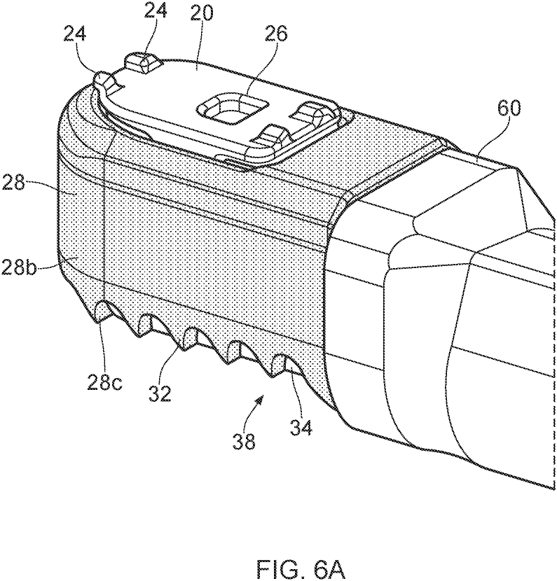

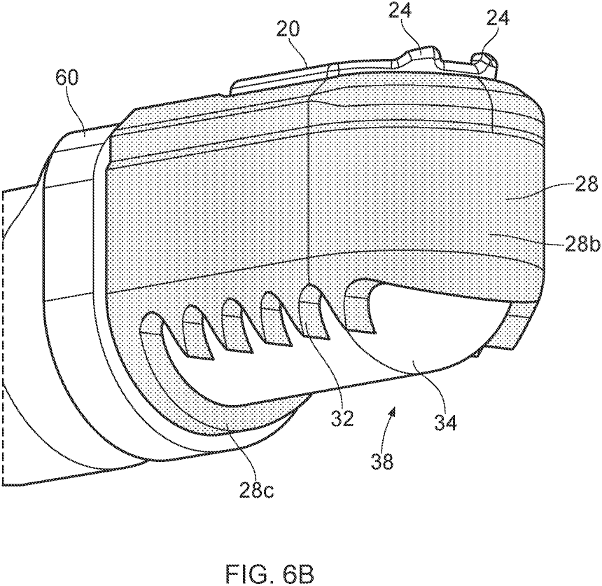

FIGS. 6A-6B show another example of a distal end portion of an electrosurgical instrument 3 having a stator 28 with a non-conductive coating, according to an embodiment of the present invention. In this embodiment, the stator 28 is entirely coated in the non-conductive material, such that the entire surface of the stator 28 is non-conductive. In particular, the entire external surface 28b is coated, the edge 28c is coated, and the inner surface (not shown) is coated in the non-conductive material. In one example, this can be achieved by omitting the masking step in the above described manufacturing method. In this embodiment, the less preferred current paths cc1 and cc2 are avoided. However, the external current path bb between the active tip and the stator 28 is also no longer present, since the entire surface of the stator 28 is non-conductive. Therefore, the stator 28 can no longer provide the return electrode. Instead, in this embodiment, the outer shaft 60 can form the return electrode since it is made of a conductive material. If the outer shaft 60 is wrapped or coated in an insulating material, a section of the shaft near or proximal to the active tip 20 can be left unwrapped / uncoated, thus exposing the conductive material of the outer shaft 60 to form the return electrode. As such, in this embodiment, the RF tracking path will be from the active tip 20 directly to the outer shaft 60, which may transport the electrical current to the generator 1 (e.g. via the cord 4 shown in FIG. 1). This embodiment still achieves the advantages of the present invention, since the undesired internal tracking paths to the internal surfaces of the stator 28 are avoided. However, this embodiment may have the further advantage of being easier to manufacture, since masking is not required.

It will be appreciated that use of the non-conductive coating is only one example way of providing a stator 28 with conductive and non-conductive areas as described above. As such, any other techniques can be employed to provide a stator that has non-conductive areas and conductive areas to prevent the internal tracking paths cc2/cc1 and to provide a return electrode as described above. For example, the stator 28 can be formed of multiple sub-components formed of different materials. The non-conductive parts of the stator 28 can be formed of one or more non-conductive subcomponents and the conductive parts of the stator 28 can be formed of one or more conductive subcomponents. Moreover, where the stator is to be entirely non-conductive (e.g. where the outer shaft 60 is the return electrode), the stator can be made entirely non-conductive using any other technique. For example, the stator can be formed of a non-conductive material such as a ceramic, in which case the non-conductive coating can be omitted.

Various further modifications to the above described embodiments, whether by way of addition, deletion or substitution, will be apparent to the skilled person to provide additional embodiments, any and all of which are intended to be encompassed by the appended claims.

1. An end effector for an electrosurgical instrument, comprising:
an electrode assembly including an active electrode for supplying radio-frequency (RF) power to a surgical site; and
a rotary shaver arrangement comprising:
a rotatable shaver blade, and
a stator that partially surrounds the rotatable shaver blade, wherein at least part of the stator is electrically non-conductive.

2. The end effector of claim 1, wherein:
the active electrode comprises an aperture which provides access to a lumen for carrying fluid from the surgical site,
the lumen is at least in part defined by an inner surface of the stator, and
at least the inner surface of the stator is non-conductive.

3. The end effector of claim 1, wherein the stator comprises an outer surface, and at least part of the outer surface of the stator is electrically conductive to form a return electrode of the electrode assembly.

4. The end effector of claim 1, wherein the active electrode is coupled to a coupling region of the stator, wherein the coupling region is non-conductive.

5. The end effector of claim 1, wherein there is no ceramic or polymer insulating element in between the active electrode and the stator.

6. The end effector of claim 1, further comprising an insulating element provided in between the active electrode and the stator.

7. The end effector of claim 3, further comprising a retention means for retaining the active electrode to the coupling region or to the insulating element.

8. The end effector of claim 1, wherein the stator comprises:
a substrate formed of a conductive material; and
a non-conductive coating provided over at least part of the substrate.

9. The end effector of claim 8, wherein the inner surface of the stator is non-conductive by means of the non-conductive coating.

10. The end effector of claim 8, wherein at least part of the outer surface of the stator is conductive by an absence of the non-conductive coating.

11. The end effector of claim 8, wherein the coupling region is non-conductive by means of the non-conductive coating.

12. The end effector of claim 8, wherein the non-conductive coating is a diamond-like carbon (DLC).

13. The end effector of claim 8, wherein the substrate of the stator is formed from a metal, and preferably wherein the metal is any one of copper, stainless steel, tungsten or an alloy of tungsten and platinum.

14. The end effector of claim 1, the stator comprising an outer surface, wherein the outer surface is non-conductive, optionally wherein the entire surface of the stator is non-conductive.

15. The end effector of claim 14, further comprising an outer shaft, wherein the outer shaft is electrically conductive to form a return electrode of the electrode assembly.

16. The end effector of claim 15, wherein the outer shaft is at least partially covered in an insulating material, optionally wherein the insulating material is a heat-shrink.

17. The end effector of claim 14, wherein the inner surface and the outer surface of the stator are non-conductive by means of a non-conductive coating, optionally wherein the entire surface of the stator is coated in the non-conductive coating.

18. The end effector of claim 1, wherein the rotatable shaver blade is formed from a non-conductive material, preferably a ceramic or insulated steel.

19. An electrosurgical instrument, comprising:
a hand-piece;
one or more user-operable buttons on the handpiece for operably controlling the instrument, and
an operative shaft, having RF electrical connections, and drive componentry for an end effector, the electrosurgical instrument further comprising an end effector, comprising:
i) an electrode assembly including an active electrode for supplying radio-frequency (RF) power to a surgical site; and
ii) a rotary shaver arrangement comprising a rotatable shaver blade, and a stator that partially surrounds the rotatable shaver blade, wherein at least part of the stator is electrically non-conductive;
the electrosurgical instrument being further arranged such that the rotary shaver arrangement of the end effector is operably connected to the drive componentry to drive the rotary shaver arrangement to operate in use, and the active electrode is connected to at least one of the RF electrical connections.

20. An electrosurgical system, comprising:
an RF electrosurgical generator;
a suction source; and
an electrosurgical instrument, the electrosurgical instrument comprising: a hand-piece; one or more user-operable buttons on the handpiece for operably controlling the instrument; an operative shaft, having RF electrical connections and drive componentry for an end effector; and an end effector, the end effector comprising:
i) an electrode assembly including an active electrode for supplying radio-frequency (RF) power to a surgical site; and
ii) a rotary shaver arrangement comprising a rotatable shaver blade, and a stator that partially surrounds the rotatable shaver blade, wherein at least part of the stator is electrically non-conductive;
the electrosurgical instrument being further arranged such that the rotary shaver arrangement of the end effector is operably connected to the drive componentry to drive the rotary shaver arrangement to operate in use, and the active electrode is connected to at least one of the RF electrical connections;
the electrosurgical instrument being further arranged such that in use the RF electrosurgical generator supplies an RF signal having a coagulation or ablation waveform via the RF electrical connections to the active electrode of the end effector.
